(19) United States
(12) Patent Application Publication
(10) Pub. No.: US 2003/0093060 A1
(43) Pub. Date: May 15, 2003

(54) Catheter assembly

(75) Inventor: Mark A. Kempf, Inver Grove Heights, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Vadnais Technologies Corporation, Saint Paul, MN

(21) Appl. No.: 10/007,537

(22) Filed: Nov. 9, 2001

(51) Int. Cl. A61M 25/00

(52) U.S. Cl. 604/527

(57) ABSTRACT

A catheter assembly defines a lumen. The catheter assembly includes a tight wound spring coil comprised of a lubricious coil material wherein the spring coil defines the lumen and has an outer surface. A wrap is wound about the outer surface of the spring coil. An exterior coating covers the spring coil and the wrap.

### CROSS-REFERENCE TO RELATED APPLICATION(S)

None.

### BACKGROUND OF THE INVENTION

The present invention relates to a catheter assembly and more particularly, the invention relates to an improved lumen-defining catheter assembly for receiving a guide wire or another component.

Catheters are used to reach remote regions of the human body for various medical reasons, including diagnostic and therapeutic procedures. Catheters are typically used within the circulatory system, the neurological system, or the urinary system to access difficult to reach sites within the body. The size and location of these sites within the body makes size, steerability and flexibility important characteristics in the catheter design.

Catheters are typically introduced into the body through a large artery and then guided through increasingly narrow regions and blood vessels until the catheter reaches the desired location. Some catheters use a guide wire to navigate the circulatory system for the catheter. The guide wire is maneuvered through the proper path in the body and the catheter (which has a lumen) is then slid over the guide wire so the catheter may reach the remote region of the body. Guide wires are typically more flexible and agile than a catheter and therefore are easier to navigate through the narrow and curvy systems of the body. The catheter fits over the guide wire and slides along the guide wire, with the guide wire located in the lumen of the catheter. Sometimes another component, such as a second catheter, is passed through the first catheter to deliver fluids or aid in performing procedures at remote regions of the body.

Catheter assemblies generally define at least one lumen, or passageway, for receiving the guide wire or other components. Dilatation catheters may also include an inner member which defines the lumen. To aid the catheter in sliding along the guide wire, a lubricious coating may be added to the guide wire or a lubricious liner (such as a Teflon™ liner) may be used within the lumen of the catheter. However, adding a lubricious coating to the guide wire increases manufacturing costs and time. Additionally, the coating prevents standard guide wires currently available on the market from being used with a variety of catheter assemblies.

It is also desirable for catheters to reach even more remote regions within the human body, thereby requiring increasingly smaller diameters of the catheter. A lumen liner typically has a thickness between about 0.001″ and about 0.002″, which takes up a significant amount of space within the catheter design. Elimination of the liner while maintaining lubricity within the lumen of the catheter would result in the ability to manufacture a significantly smaller catheter which is able to reach more remote regions of the human body, thereby gaining a competitive advantage for the manufacturer.

Once the catheter is slid over the guide wire, steerability and flexibility are important for maneuvering the catheter along the guide wire. When the catheter is inserted into a body, an operator manipulates a proximal end of the catheter to maneuver the catheter along the guide wire and through the body. The catheter assembly defines the stiffness and flexibility of the catheter. The catheter must be stiff enough to allow pushing, pulling and manipulation of the catheter, yet flexible enough to permit passage through increasingly smaller blood vessels and the curvy circulatory system.

There is a need in the art for a catheter having an increasingly smaller diameter that allows easy passage through the lumen (for a guide wire or other component), and improved steerability and flexibility.

### BRIEF SUMMARY OF THE INVENTION

The present invention relates to a catheter assembly defining a lumen. The catheter assembly includes a tight wound spring coil comprised of a coil material wherein the spring coil defines the lumen and has an outer surface. In one embodiment, a wrap is wound around the outer surface of the spring coil. A polymer based coating covers the spring coil or the spring coil and the wrap.

### BRIEF DESCRIPTION OF THE DRAWINGS

FIG. 1 is a view of a catheter.

FIGS. 2A through 2E is a schematic view of the method for fabricating a catheter assembly of the present invention.

FIG. 3 is a cutaway view of the catheter assembly shown in FIG. 2E.

FIG. 4 is a cross-sectional view of the catheter assembly shown in FIG. 2E.

FIG. 5 is a sectional view of the catheter assembly shown in FIG. 2E taken substantially along the longitudinal axis.

### DETAILED DESCRIPTION

The present invention relates to a lumen-defining member. In particular, the present invention lumen-defining member may comprise an entire catheter or a component to be received within the catheter, such as an inner member or a guide wire. In the following discussion, the present invention lumen-defining member will be discussed with respect to a catheter assembly defining a lumen for receiving a guide wire. Those skilled in the art will recognize the structure and the method of fabrication disclosed for the catheter assembly is applicable to other catheter components for defining a lumen. For example, the lumen-defining member may be an inner member of a catheter, a catheter, an assembly attached to a hypotube, or a rapid exchange catheter. The present invention is also applicable to micro-catheters for neurological and cardiac intervention and drug delivery and other technologies, such as inner members and outer members for PTCA catheters, catheters for deployment of coils for neurological intervention procedures, electro-physiology catheters and lead placement catheters.

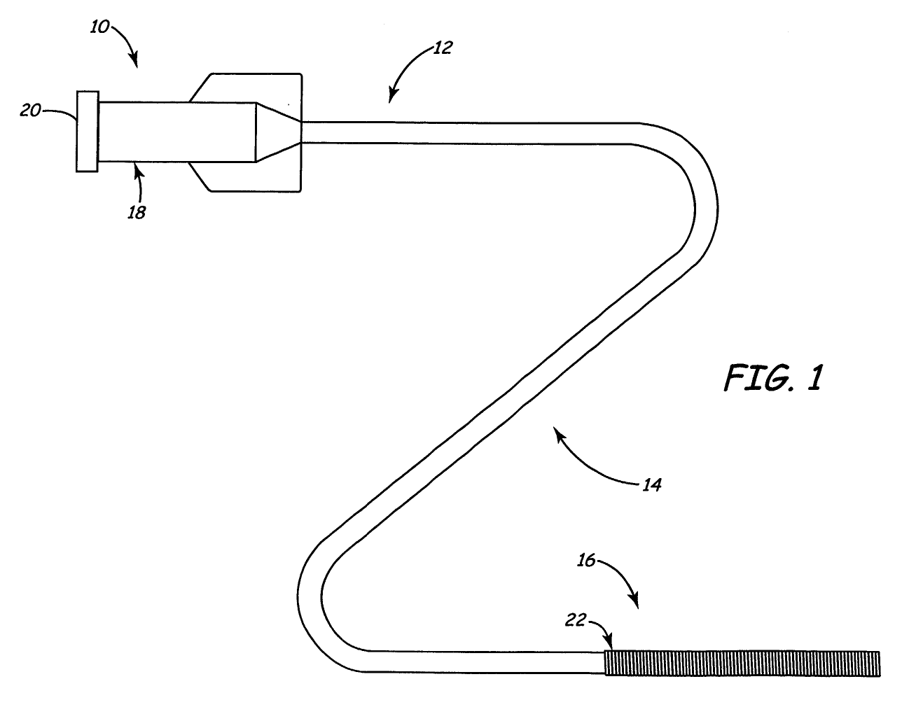

FIG. 1 is a view of a catheter 10. The catheter 10 is typically comprised of three sections, a proximal section 12, an intermediate section 14 and a distal section 16. Typically the proximal section 12 includes a control means 18. The control means 18 provides access to a lumen (not shown) for inserting a guide wire (not shown), and provides fluid access to the catheter 10. The guide wire is preferably inserted into the body through a large artery and then guided through increasingly narrow regions and blood vessels until it reaches the desired location. Catheter 10 is then slid over the guide wire until the distal section 16 of the catheter 10 reaches the desired location. The control means 18 are located at the proximal end of the catheter 10 to steer the catheter 10 through the body. An opening 20 or port is also located in the control means 18, as well as a seal, to allow fluid to be directed toward the distal end of the catheter 10. The catheter 10 has a small outer diameter, preferably between about 18 mil and about 21.5 mil, so that the catheter 10 is able to reach increasingly remote regions within the body. A portion of the distal section 16 of the catheter 10 shown in FIG. 1 is cutaway to show a portion of a catheter assembly 22 (the portion of the catheter 10 inserted into a body) which defines a lumen, or passageway, for receiving the guide wire.

The distal section 16 of the catheter 10 is typically introduced into the body through a large artery and then guided through increasingly narrow regions of the circulatory system until the catheter 10 reaches the desired location. The catheter 10 may also be used to access remote regions of the neurological and urinary systems. The guide wire is first inserted to navigate the blood vessels and other difficult to maneuver regions of the body. Control means 18 steer the guide wire through the body. The catheter assembly 22 is slid over and along the guide wire so the catheter 10 can access remote regions of the body. In some applications, the guide wire can be removed from the lumen of catheter assembly 22 after the catheter assembly 22 is placed, so that the lumen can be used to deliver drugs and other devices through the lumen.

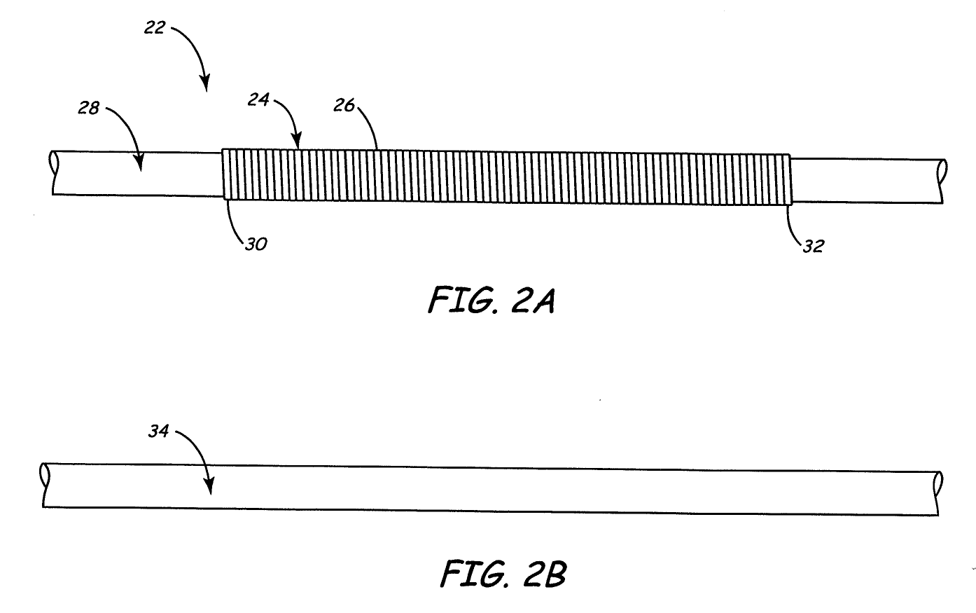

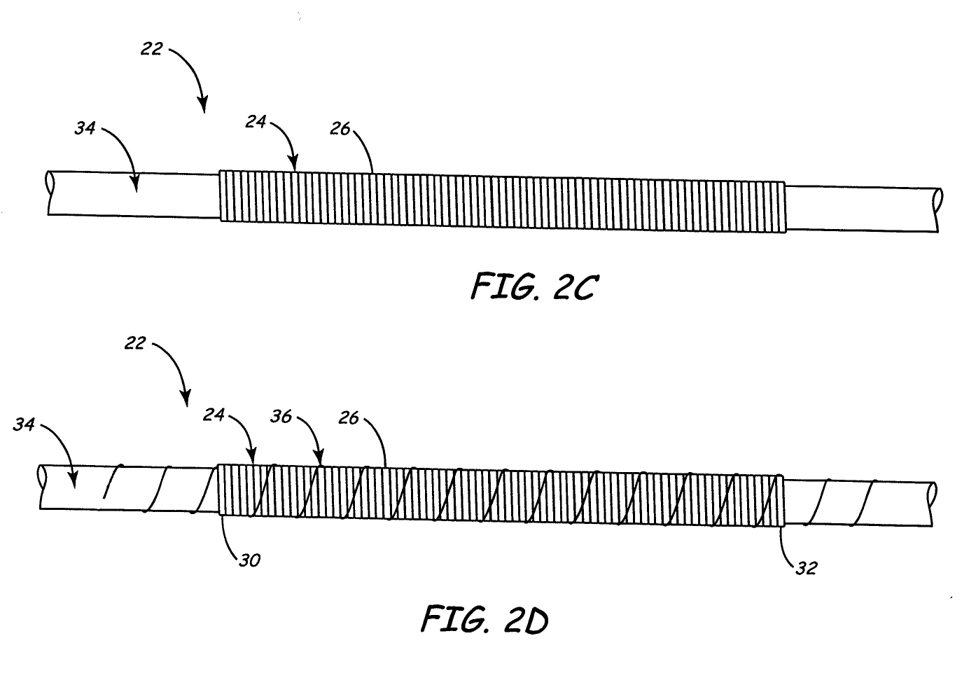

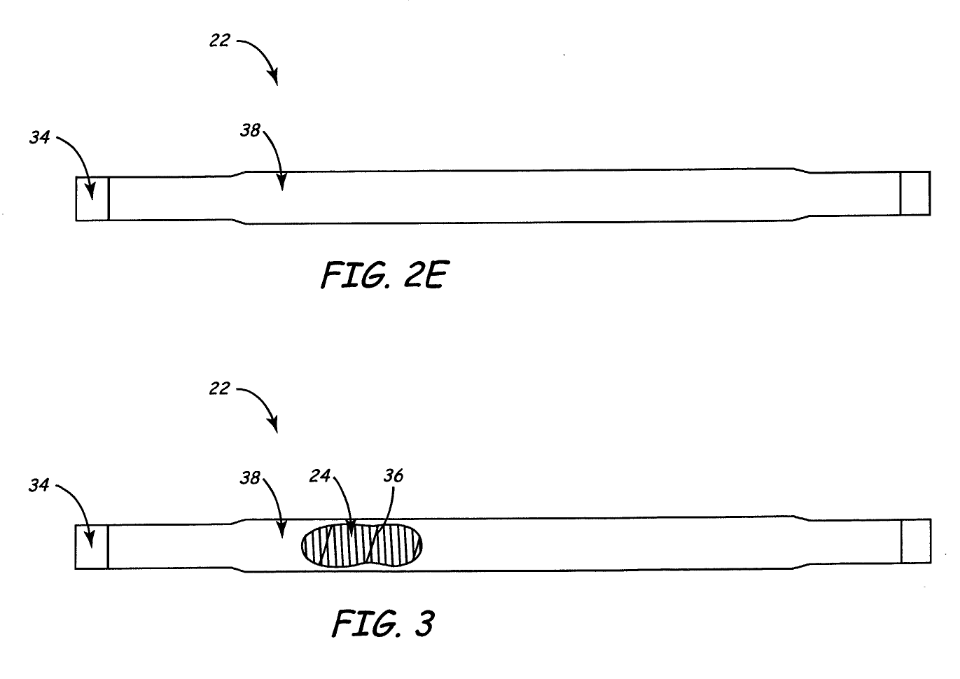

FIGS. 2A through 2E shows schematic views of a method for fabricating the catheter assembly 22. As seen in FIG. 2A, a spring coil 24 is formed by tightly winding a lubricious coil material 26 about a first mandrel 28 having a diameter smaller than a diameter of the desired guide wire. The coil material 26 of the spring coil 24 is preferably wound about the first mandrel 28 from a distal end 30 to a proximal end 32. Once the coil material 26 is wound, the spring coil 24 is removed from the first mandrel 28 and the spring coil 24 expands to its equilibrium state. The inner diameter of the spring coil 24, in its equilibrium state, defines the lumen, or passageway, for receiving the guide wire.

A second mandrel 34 is shown in FIG. 2B. The second mandrel 34 has a diameter larger than a diameter of the first mandrel 28 and will generally fill the entire lumen of coil 24 once the spring coil 24 expands to its equilibrium state. A diameter of the desired guide wire will typically have a diameter greater than the diameter of the first mandrel 28 and smaller than the diameter of the second mandrel 34. As seen in FIG. 2C, the second mandrel 34 is inserted into the lumen of the spring coil 24. The spring coil 24 forms an inner layer of the catheter assembly 22. The spring coil 24 is comprised of the lubricious coil material 26, which is preferably a spring wire coated with a hydrophilic polymeric material. Alternatively, the spring wire maybe coated with a polytetrafluoroethylene (PTFE) such as Teflon™. The hydrophilic coating mixes easily with water and acts as a lubricant so that the spring coil 24, and thus the catheter 10, slides easily along the guide wire and is easily maneuverable over the guide wire or other component for use with the catheter. The spring wire is preferably comprised of SS304V, although stainless steel alloys, nickel titanium alloys, titanium alloys, Tungsten, platinum and its alloys, or MP35N, or other spring materials may be used for the spring wire.

The hydrophilic polymeric coating used to pre-coat the spring wire is low friction. The coating is applied to the spring wire before it is used to fabricate the catheter assembly 22. In the past, catheters used lumen liners to increase lubricity of the inner lumen. The liner had a thickness between about 0.7 mil and about 2.0 mil. The hydrophilic coating on the spring wire eliminates the need for a lumen liner within the catheter 10. Eliminating the use of a liner reduces the diameter while maintaining flexibility of the catheter 10. As discussed below, the catheter assembly 22 utilizing the lubricious coil material has a diameter smaller than the diameter of the catheter assembly 22 with the spring coil 24 and a lumen liner. Thus, the catheter 10 is made with a smaller diameter without a loss of lubricity or flexibility. Alternatively, the coating may be applied to one side of the spring wire, rather than the entire diameter, to further reduce the diameter of the catheter 10.

The spring coil 24 has high axial stiffness which is helpful for pushing the catheter 10 along the guide wire and maneuvering the catheter 10 through the increasingly narrow and curvy regions of the body. Preferably, the spring coil 24 is tight wound and there is no spacing, or a spacing less than a width of the coil material 26, between windings. A tight wound coil 24 allows significant force to be applied to the coil 24 in order to push the coil 24 to a desired location within the body, without significantly compressing or distorting the coil 24.

In an alternative embodiment of the catheter 10, the spacing between windings of the spring coil 24 maybe greater than the width of the coil material 26, that is the spring coil 24 is open wound.

As seen in FIG. 2D, once the second mandrel 34 is positioned within the spring coil 24, a wrap 36 is wound about an outer surface of the spring coil 24. The wrap 36 forms an outer layer of the catheter assembly 22. The wrap 36 is preferably wound in a direction counter to the windings of the spring coil 24, from the proximal end 32 to the distal end 30 of the spring coil 24. The wrap 36 is preferably comprised of a polymer based thread, such as Vectran™ or Dyneema™. The polymer based thread has a high pitch counterwind and high tensile strength. The counterwind and the high tensile strength aids in the pull and torsion capabilities of the catheter 10. Preferably, the wrap 36 has about one winding for about every 20 to 30 windings of the spring coil 24. The wrap 36 has an open wind such that windings of the wrap 36 have a pitch greater than a width of the fiber. Also, the pitch of the wrap 36 is preferably greater than the pitch of the spring coil 24. The wrap 36 extends past the proximal and distal ends 32 and 30 of the spring coil 24 and helps to contain the spring coil 24. The ends of the wrap 36 are glued to the second mandrel 34 past the spring coil 24. In further embodiments of the present invention, the wrap 36 may have a like wind to the windings of the spring coil 24 or the wrap may have a tight wind about the spring coil 24. Further embodiments of the catheter assembly 22 may not include the wrap 36.

After the wrap 36 is wound about the spring coil 24, an exterior coating 38 is applied to the spring coil 24 and the wrap 36, as seen in FIG. 2E. FIG. 3 shows a cutaway view of the catheter assembly of FIG. 2E, revealing the spring coil 24 and the wrap 36 within the exterior coating 38. The exterior coating 38, in association with the wrap 36, helps to contain the spring coil 24 and the wrap 36 to hold the catheter assembly 22 together. The exterior coating 38 provides an air and liquid tight seal to protect the spring coil 24 and the wrap 36. After the exterior coating 38 is applied to the spring coil 24 and the wrap 36, the ends of the catheter assembly 22 beyond the coil 24 is cut off to remove the glued portions of the wrap 36. The second mandrel 34 is then removed from the lumen. Typically, the catheter assembly 22 is kept hollow until the end user inserts the guide wire or other applicable component, if desired.

The exterior coating 38 is preferably comprised of a low friction polymeric or flouropolymeric material, such as PEBAX, Nylon™, PE or Teflon™. Alternatively, the exterior coating 38 maybe an extrusion. The exterior coating 38 also provides lubrication to the outer surface of the catheter assembly 22. The type of polymer used for the exterior coating 38 may vary along the length of the catheter 10 such that the flexibility of the catheter 10 is varied along its length. The exterior coating 38 forms a more consistent, smooth and constant layer for the outer surface of the catheter assembly 22. Additionally, an adhesive may be used to secure the spring coil 24 and the wrap 36 to the exterior coating 38. Examples of an adhesive to be used include a UV adhesive, heat activated or non-heat activated adhesive, or an adhesive activated by a lack of oxygen.

The catheter assembly 22, as seen in FIG. 3, is stiff enough to allow pushing, pulling and manipulation of the catheter 10, yet flexible enough to permit passage of the catheter 10 through increasingly smaller and curvy circulatory or neurological system. The tight wound spring coil 24 allows significant force to be applied to the coil 24 in order to push the catheter to a desired location within the body without significantly compressing or distorting the coil 24. Counterwinding the wrap 36 adds strength to the catheter assembly 22 for pulling and twisting the catheter without distorting the catheter assembly 22 and keeping the catheter assembly 22 together.

In further embodiments of the present invention, the flexibility of the catheter may be varied along its length. In particular, it is desirable for the catheter to be more flexible at its distal end than at its proximal end. One method of varying the flexibility of the catheter is to vary an initial tension of the spring coil 24 along a length of the spring coil 24. The initial tension refers to the tension required to cause separation between windings of the spring coil 24. By winding the spring coil 24 with a low initial tension in its distal end and with a higher initial tension at its proximal end, desirable characteristics of the catheter 10 may be achieved.

Another method for varying the flexibility of the catheter along its length is to vary the durometer of the exterior coating 38, that is, vary the measure of flexibility of the material of the exterior coating 38 along the length of the catheter 10. In addition, varying the thickness or concentration of the exterior coating 38 varies the flexibility of the catheter along its length. For example, the thickness of the exterior coating 38 would be thicker at the proximal section and thinner at the distal section to provide more flexibility at the distal end.

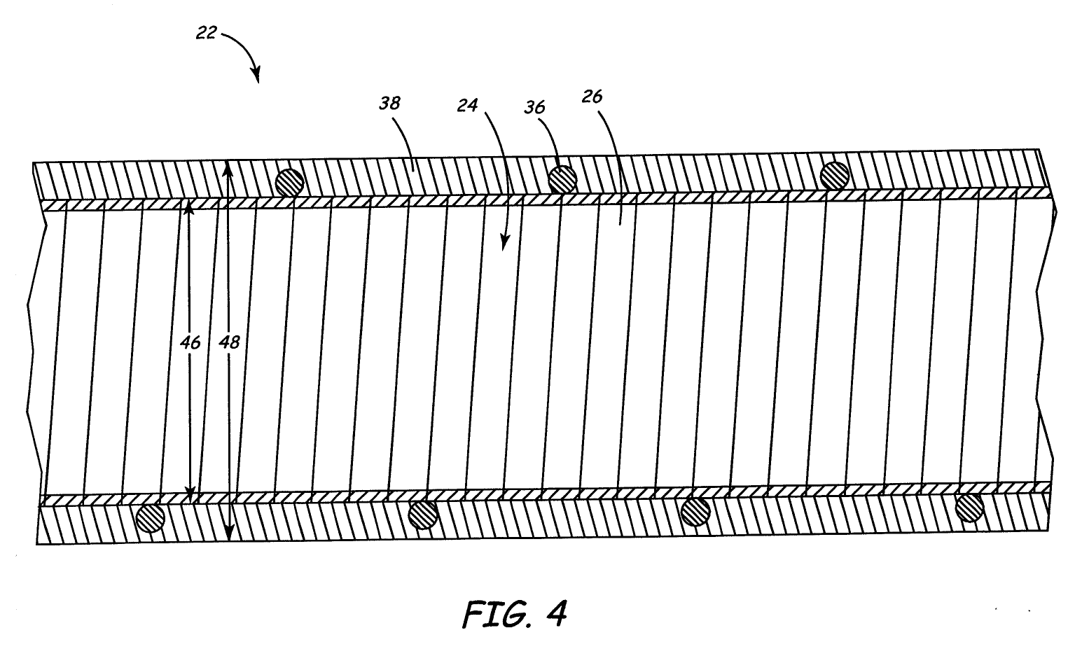

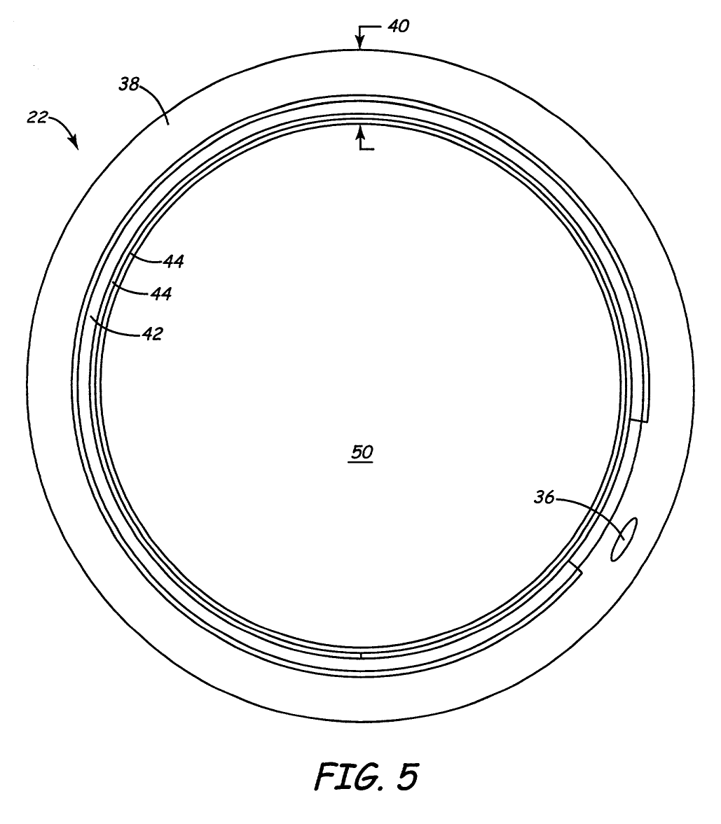

FIG. 4 is a sectional view of the catheter assembly 22 taken along a longitudinal axis and FIG. 5 is a cross-sectional view of the catheter assembly 22. The catheter assembly is comprised of the spring coil 24, the wrap 36 wound about the spring coil 24 and the exterior coating 38 encasing both the spring coil 24 and the wrap 36. The spring coil 24 is comprised of a spring wire 42 coated on all sides with a hydrophilic polymer material 44.

The catheter assembly 22 preferably has a thickness 40 between about 2.2 mil and about 3.1 mil. The thickness 40 includes the spring coil 24 (the spring wire 42 and polymer coating 44), the wrap 36 and the exterior coating 38. Preferably, the coil material 26 (forming the spring coil 24) is about 0.9 mil thick. The spring wire 42 has a thickness of about 0.7 mil and a width of about 0.3 mil, and the polymer material 44 is about 0.1 mil thick on each side of the spring wire 42. The polymer based fiber wrap 36 is between about 0.5 mil to about 1.0 mil, and most preferably about 0.5 mil thick. The exterior coating 38 is about 1 mil thick. An inner diameter 46 of the catheter assembly 22 is about 16 mil. An outer diameter 48 of the catheter assembly 22, and thereby the catheter 10 is preferably between about 18 mil and about 21.5 mil. Preferably, the diameter of a guide wire 50 is less than the inner diameter 46 of the catheter assembly 22.

The lumen-defining member for a catheter of the present invention provides a catheter having an increasingly smaller diameter and allows easy passage through the lumen of catheter components. Although discussed with respect to a catheter assembly, the lumen-defining member may comprise different embodiments and components for a variety of catheters. For example, the lumen-defining member may be catheter or inner member for a guide catheter, such as for use in a neurological system. Additionally, the lumen-defining member may be an inner or outer member for a dilatation catheter or a stent delivery catheter. The lumen-defining member may also define a catheter for use with or without a guide wire, or an inner member for attachment to a hypotube within a catheter.

In an alternative embodiment of the present invention, the lumen-defining member is an inner member for insertion within the catheter 10. The inner member defines a lumen for receiving the guide wire, or other catheter components. The inner member embodiment can be used in a balloon catheter, which includes an inflatable balloon. The guide wire is inserted into the body to navigate the circulatory, neurological or urinary system until it reaches the desired region of the body. The inner member, and thereby the catheter, is slid over and along the guide wire until the catheter reaches its desired location.

In use in a balloon catheter, the inner member can withstand external pressure created by the blowing up of the balloon. A balloon catheter includes the balloon at the distal end that is inflated to open blocked arteries. Air is blown through the catheter to inflate the balloon. The inner member is also located within the catheter. The tight wound coil of the inner member provides a good source of resistance to external pressures exerted on the outer surface of the inner member during inflation of the balloon.

Although the present invention has been described with reference to preferred embodiments, workers skilled in the art will recognize that changes may be made in form and detail without departing from the spirit and scope of the invention. For example, the present invention lumen-defining member is primarily discussed with respect to a catheter. The present invention is applicable to the lumen-defining member may be an inner member of a catheter, a catheter, an assembly attached to a hypotube, or a rapid exchange catheter. The present invention is also applicable to micro-catheters for neurological and cardiac intervention and drug delivery and other technologies, such as inner members and outer members for PTCA catheters, catheters for deployment of coils for neurological intervention procedures, electro-physiology catheters and lead placement catheters.

1. A catheter assembly defining a lumen, the catheter assembly comprising:
a tight wound spring coil comprised of a coil material wherein the spring coil defines the lumen and has an outer surface;
a wrap wound about the outer surface of the spring coil; and
an exterior coating covering the spring coil and the wrap.

2. The catheter assembly of claim 1 wherein the coil material is lubricious and comprised of a spring wire coated with a hydrophilic polymeric material.

3. The catheter assembly of claim 2 wherein the spring wire is coated by a PTFE material.

4. The catheter assembly of claim 1 wherein the wrap is comprised of a polymer based thread.

5. The catheter assembly of claim 4 wherein the polymer based thread is comprised of Vectran™.

6. The catheter assembly of claim 4 wherein the polymer based thread is comprised of Dyneema™.

7. The catheter assembly of claim 1 wherein windings of the spring coil have a pitch greater than a width of the coil material.

8. The catheter assembly of claim 1 wherein an initial tension of the spring coil varies along a length of the spring coil.

9. The catheter assembly of claim 1 wherein the wrap is tight wound.

10. The catheter assembly of claim 1 wherein the wrap is wound about the outer surface of the spring coil in a direction counter to windings of the spring coil

11. The catheter assembly of claim 1 wherein a ratio of windings of the wrap to the windings of the spring coil is about 1 to between about 20 to 30.

12. The catheter assembly of claim 1 wherein the spring coil has a proximal end and a distal end, and the wrap extends beyond and secures the proximal and distal ends of the spring coil.

13. The catheter assembly of claim 1 wherein an adhesive secures the spring coil and the wrap to the exterior coating.

14. The catheter assembly of claim 1 wherein the catheter assembly has a thickness between about 1.8 mils and about 3.2 mils.

15. The catheter assembly of claim 1 wherein the spring coil and the wrap are in an equilibrium state within the exterior coating.

16. A lumen-defining component for a catheter assembly, the component comprising:
a lubricious spring coil defining a lumen, the spring coil having a proximal end, a distal end and an outer surface wherein the spring coil is tight wound;
a wrap wound about the outer surface of the spring coil; and
an exterior coating covering the spring coil and the wrap.

17. The component of claim 16 wherein the spring coil is comprised of a spring wire coated with a hydrophilic polymeric material.

18. The component of claim 17 wherein the spring coil is coated with a PTFE material.

19. The component of claim 16 wherein an initial tension of the spring coil varies along a length of the spring coil.

20. The component of claim 16 wherein the wrap is comprised of a polymer based thread.

21. The component of claim 16 wherein the wrap is wound in a direction counter to windings of the spring coil.

22. The component of claim 16 wherein the component has a thickness between about 1.8 mils and about 3.2 mils.

23. The component of claim 16 wherein a ratio of windings of the wrap to windings of the spring coil is one to between about 20 to 30.

24. The component of claim 16 wherein the spring coil is open wound.

25. The component of claim 16 wherein the wrap is open wound.

26. The component of claim 16 wherein the spring coil and the wrap are in an equilibrium state within the exterior coating.

27. A catheter assembly comprising:
a tight wound spring coil comprised of a pre-coated coil material wherein the spring coil defines a lumen and the spring coil has an outer surface; and
an exterior coating covering the outer surface of the spring coil.

28. The catheter assembly of claim 27 wherein the spring coil has an inner diameter of about 16 mils.

29. The catheter assembly of claim 27 wherein the pre-coated coil material is comprised of a spring wire coated with a hydrophilic polymeric material.

30. The catheter assembly of claim 27 wherein an adhesive secures the spring coil to the exterior coating.

31. The catheter assembly of claim 27 wherein windings of the spring coil have a pitch greater than a width of the coil material.

32. The catheter assembly of claim 27, and further comprising a polymer based thread wound about the outer surface of the spring coil.

33. The catheter assembly of claim 32 wherein the thread is wound about the spring coil in a direction counter to windings of the spring coil.

34. The catheter assembly of claim 32 wherein the catheter assembly has a thickness of about 1.8 mils to about 3.2 mils.

35. The catheter assembly of claim 27 wherein an initial tension of the spring coil varies along a length of the spring coil.

36. A method for forming a catheter assembly, the method comprising:
winding a lubricious coil material around a first mandrel to form a spring coil wherein the spring coil has an outer surface;
removing the spring coil from the first mandrel wherein the spring coil expands to an equilibrium state and defines a lumen having an inner diameter;
winding adheres to mandrel a wrap about the outer surface of the spring coil; and
covering the spring coil and the wrap with an exterior coating.

37. The method of claim 36, and further comprising inserting a second mandrel into the lumen after removing the spring coil from the first mandrel wherein the second mandrel has a diameter greater than that of the first mandrel.

38. The method of claim 36 wherein the wrap is wound about the spring coil in a direction counter to windings of the spring coil.

39. The method of claim 36 wherein the spring coil has a proximal end and a distal end, the wrap extending beyond and securing the proximal and distal ends of the spring coil.

40. The method of claim 36 wherein the lubricious coil material comprises a spring coil coated with a hydrophilic polymeric material.

41. The method of claim 36 wherein the catheter assembly has a thickness between about 1.8 mils and about 3.2 mils.

42. The method of claim 36 wherein a ratio of windings of the wrap to the windings of the spring coil is about 1 to between about 20 to 30.

43. The method of claim 36 wherein the wrap is comprised of a polymer based thread.

44. A method for forming a catheter assembly, the method comprising:
winding a pre-coated coil material around a first mandrel to form a spring coil having a proximal end, a distal end and an outer surface;
removing the spring coil from the first mandrel wherein the spring coil expands to an equilibrium state and defines a lumen having an inner diameter; and
covering the spring coil with an exterior coating.

45. The method of claim 44, and further comprising using an adhesive to secure the spring coil to the exterior coating.

46. The method of claim 44 wherein the pre-coated coil material is comprised of a spring wire coated with a hydrophilic polymeric material.

47. The method of claim 44 and further comprising inserting a second mandrel into the lumen wherein the second mandrel has a diameter greater than that of the first mandrel.

48. The method of claim 44, and further comprising varying an initial tension of the spring coil along a length of the spring coil.

49. The method of claim 44, and further comprising:
winding a polymer based thread about the outer surface of the spring coil; and
adhering the polymer based thread to the spring coil.

50. The method of claim 49 wherein the polymer based thread is wound about the spring coil in a direction counter to windings of the spring coil.
